(19) United States
(12) Patent Application Publication
(10) Pub. No.: US 2012/0237582 A1
(43) Pub. Date: Sep. 20, 2012

(54) METHOD FOR MANUFACTURING A SILICA POWDER COATED WITH AN ANTIBACTERIAL AGENT, AND TOPICAL DERMATOLOGICAL COMPOSITION INCLUDING THE SAME

(75) Inventors: Youn-kyung Do, Incheon (KR); Yun-jeong Kim, Wonju-si (KR); Ji-hyun Son, Seoul (KR); Hyun-sang Lee, Suwon-si (KR); Jong-woo Cheon, Seongnam-si (KR)

(73) Assignee: ACT CO., LTD., Eumseong-gun, Chungcheongbuk-do (KR)

(21) Appl. No.: 13/512,837

(22) PCT Filed: Dec. 11, 2009
(86) PCT No.: PCT/KR2009/007449

§ 371 Date: May 30, 2012

(30) Foreign Application Priority Data
Dec. 1, 2009 (KR) 10-2009-0117593

(51) Int. Cl. A61K 8/02; A61Q 19/00; B05D 5/00; B82Y 40/00; B82Y 30/00

(52) U.S. Cl. 424/401; 427/215; 977/775; 977/810; 977/926; 977/890

(57) ABSTRACT

Provided are a method of preparing a silica powder coated with an antibacterial agent and a topical dermatological composition including the silica powder, and more particularly, a method of preparing a silica powder coated with an antibacterial agent by forming a silica particle by inducing a reaction between silicon alkoxide and alcohol solvent in the presence of a catalyst and then forming an silver and metallic coating layer on the silica particle, and a method of preparing a topical dermatological composition including the silica powder. The silica powder coated with the antibacterial agent may have high antibacterial capability, and thus, topical dermatological products including the silica powder may be maintained for a long time without using a chemical antiseptic agent that may irritate the skin and cause an allergy in the human body. Even if a small amount of expensive silver (Ag) ion material is used, the silica powder has excellent antibacterial capability and stability. In addition, since the silica powder exhibits bright color, colors may be freely expressed when used in a composition of cosmetics, and thus, there is no is limit in using the silica powder in bright-color cosmetics as well as in dark-color cosmetics.

### TECHNICAL FIELD

The present invention relates to a method of manufacturing a silica powder coated with an antibacterial agent and a topical dermatological composition including the silica powder, and more particularly, to a method of manufacturing a silica powder coated with an antibacterial agent by including forming a silica particle by inducing a reaction between silicon alkoxide and an alcohol solvent in the presence of a catalyst and then forming an silver (Ag) and metallic coating layer on the silica particle, and a method of manufacturing a topical dermatological composition including the silica powder.

### BACKGROUND ART

As a method of improving the properties of metal and ceramic powder materials, microminiature power materials with a size of 1 μm or less in technologies for miniaturizing particles have received considerable attention.

In general, from among microminiature power materials, a powder with a size of 0.1 μm or less is referred to as a nanoscaled powder and a powder with a size of 0.1 to 1 μm is referred to a submicron powder. As the particle size of microminiature power material becomes microminiature, unique mechanical and physical properties that are not exhibited in general powder materials are exhibited.

That is, as a particle size is reduced, a surface area is increased, volume properties are reduced, and surface properties are remarkably exhibited. In order to prepare such microminiature particles, various mechanical, physical, or chemical methods have been attempted. However, many problems arise when obtaining the selective uniformity of particle sizes and there is a technological limit in performing a mass-production process and preparing two or more composite particles.

In order to apply and use prepared microminiature particles, the regular and uniform dispersion state and oxidation stability of the microminiature particles need to be ensured.

Inorganic materials used in application fields, in particular, in topical dermatological products have been applied to various fields, for example, skin care, makeup, sunblock, ointment, powder, or the like. In this case, when inorganic materials are applied to products, inorganic materials need to be highly compatible with other components, needs not to precipitate and discolor, and needs to have unchanged smell.

In addition, when inorganic materials are applied to the skin, inorganic materials require high stability, high skin adhesion, no turbidity, and excellent antibacterial properties. However, there is a limit in satisfying these conditions by using the current technologies.

There are many kinds of antibacterial agents that have been currently used. An antibacterial agent is largely classified into an organic antibacterial agent and an inorganic antibacterial agent. Organic antibacterial agents are easily processed and do not largely affect the mechanical physical properties, transparency, color, and so on of a final product, compared to inorganic antibacterial agents. Thus, so far, methylparaben or propylparaben-based organic antibacterial agents have been largely used. However, it is has been reported that organic antibacterial agents adversely affect biological skin cells and irritate the skin.

As the stability of an organic antibacterial agent with respect to the human body has been discussed, inorganic antibacterial agents for compensating for the disadvantages of organic antibacterial agents are attracting attention.

An inorganic antibacterial agent is prepared by putting a metal ion having excellent antibacterial properties, such as silver (Ag), copper (Cu), manganese (Mn), zinc (Zn), or the like on the surface of an inorganic support such as zeolite, silica alumina, or the like. Since an inorganic antibacterial agent has a three-dimensional structure having minute pores, an inorganic antibacterial agent has a large specific surface area and excellent heat resistance.

Silicon (Si) is the second most abundant element in the earth's crust. It has been well known that silicon has no direct sterilization effect with respect to plant pathogenic fungi but increases disease resistance and stress resistance. In addition, silicon has been largely used as materials of cosmetics in order to make cosmetics soft and to obtain high spreading properties of cosmetics.

In general, most metals having toxicity with respect to microorganisms have high toxicity with respect to the human body. However, it has been reported that a few metals such as Ag, Cu, Mn, Zn, or the like have high antibacterial properties and high stability and do not harm the human body.

Silver (Ag) used as an antibacterial agent has high antimicrobial activity, no toxicity and no irritation with respect to the human body, high chemical durability, and excellent heat resistance. In addition, silver (Ag) emits Ag ions for a long time and has excellent antimicrobial durability.

Composite materials containing Ag-based particles render antibacterial properties, deodorization properties, antistatic properties, and so on to plastic materials that are widely used in products of daily life as well as in various industrial fields. So far, researches have been conducted into technologies for developing such composite materials in various countries. Outside Korea, many basic technologies for composite materials have been reported but technologies for application and products of such composite materials have been rarely reported.

In Korea, many basic researches have been conducted into methods of preparing is Ag-based particles, such as an electrochemical method, a chemical reducing method, an optical method, an ultrasonic method, a micro method, a γ-irradiation method, or the like and many research results have been reported. However, so far, a method of controlling the dispersion and oxidation stability of Ag particles and a method of synthesizing particles having selective uniformity have not yet been developed. Thus, it is difficult to synthesize Ag particles having a uniform size from miniature powder materials with a size of 1 μm or less.

As a method of preparing an antibacterial silica support, a vapor decomposition method using silicon tetrachloride (SiCl₄) (Japanese Patent Application Publication No. 62-003011), a sol-gel method using silicon alkoxide (Japanese Patent Application Publication No. 63 -166777), a method of preparing a silica support by using a neutralization reaction between alkali silicate and acid (U.S. Pat. No. 4,675,122), and the like have been known. In this case, a method of supporting an antibacterial material such as Ag, Cu, gold (Au), Zn, platinum (Pt), or the like in a prepared support has been disclosed.

However, in the vapor decomposition method, toxicity and corrosion may be generated during a reaction and pores are formed in particle surfaces only. In the sol-gel method, high purity powders may be obtained but economic problems may arise.

The method of preparing the silica support by using the neutralization reaction has been widely used due to a low cost in regard to raw materials and excellent handleability. However, in this case, since a mixture reaction between materials is performed by dropping, an alkali silicate solution needs to have a concentration of 20% or less. Since materials regionally contact each other, materials irregularly react with each other. Since a washing process using alkaline water is required for a long time in order to increase a pore volume, it takes a long time (3-4 days/batch) to perform the preparation method. Preparing costs are increased due to very low product uniformity according to each product lot. Catalyst materials may be discharged due to long-time aging and washing processes. In addition, complex processes, that is, pulverization, assembly, and so on are required in order to control the diameter and shape of catalyst.

A method of supporting an antibacterial material in a silica support prepared by using the above-described method is largely classified into an impregnation method, an ion exchange method, and a precipitation method.

The impregnation method uses a spray method, a dry evaporation method, an adsorption method, or the like as a method in which a support contacts a solution containing an antibacterial material to support the antibacterial material in the support.

The spray method is a method in which a support is put and stirred in an evaporator and a solution containing an antibacterial material is sprayed to support the antibacterial material in the support. The dry evaporation is a method in which a support is immersed in a solution containing an antibacterial material and the solution evaporates.

The ion exchange method is a method that is generally used to support an antibacterial material in silica, zeolite, alumina, or the like. When the ion exchange method is used, the antibacterial material is uniformly distributed but only the small amount of antibacterial material can be supported and it takes a very long time to support the antibacterial material.

In the above-described conventional methods of supporting an antibacterial material, the antibacterial material needs to be supported after a support is prepared. Thus, it takes a very long time to support the antibacterial material, only a small amount of antibacterial material is supported, and complex processes needs to be performed, thereby increasing manufacturing costs.

In the method of preparing the silica support by using the neutralization reaction, antibacterial silica may be prepared by injecting a material containing an antibacterial material during initial synthesis of a material. However, a considerable amount of the antibacterial material may be lost due to excessive aging and washing processes.

That is, it is not easy to prepare a highly porous silica support by using a conventional method, only a limited amount of antibacterial material can be supported, and manufacturing costs are high due to a long manufacturing period of time.

When an antibacterial material is supported in a support, since the support needs to be previously prepared, it is difficult to uniformly support the antibacterial material. In addition, the method is economically infeasible due to complex manufacturing processes.

When an antibacterial material is added during preparation of a support, some antibacterial materials may also be lost due to a long-time aging and washing processes.

An inorganic particulate material has low toxicity and stable heat resistance compared to an organic particulate material but has a unique metallic color. In addition, when an inorganic particulate material is applied to a product, an inorganic particulate is material may become grey and may not maintain antibacterial properties and dispersion properties.

Silver (Ag) ions from among inorganic antibacterial materials have been known as a metal material having excellent antibacterial properties. In this regard, there is a method of forming an Ag coating layer on a surface of a support by mixing a support and an Ag precursor in a solvent and then adding a reducer to reduce the Ag precursor.

However, an Ag precursor used in the above-described method is expensive, thereby increasing manufacturing costs of a silica nano-powder coated with silver, prepared by using the method. Thus, it is difficult to apply the method to prepare various types of formulations due to high manufacturing costs.

The silica nano-powder coated with silver, which is prepared by using the method, is a brown particle. Thus, when used as a material of cosmetics, the material of cosmetics exhibit dark. Accordingly, there is a limit in manufacturing bright cosmetics.

Accordingly, there is a need to develop an inexpensive antibacterial agent that is economically feasible by reducing manufacturing costs and also has dispersion properties, oxidation stability, skin adaptability, and high antibacterial properties. In addition, there is a need to develop a useful antibacterial agent that exhibits a bright color such that colors may be freely expressed.

### DETAILED DESCRIPTION OF THE INVENTION

Technical Problem The present invention provides a method of manufacturing a silica powder having high antibacterial properties at low manufacturing costs.

The present invention also provides a method of manufacturing a silica powder coated with an antibacterial agent, which exhibits a bright color.

The present invention also provides a topical dermatological composition including the silica powder.

### TECHNICAL SOLUTION

According to an aspect of the present invention, there is provided a method of manufacturing a silica powder coated with an antibacterial agent, the method including a first operation of forming silica particles with an average diameter of 100 nm to 150 nm by mixing 10 to 15 parts by weight of any one catalyst selected from the group consisting of aqueous ammonia, ammonium bicarbonate, and triethanolamine and 700 to 800 parts by weight of an alcohol solvent in 100 parts by weight of silicon alkoxide to prepare a resultant, and maintaining the resultant for 6 to 7 hours at a temperature of 35 to 38 ° C. to induce a reaction of the resultant; and a second operation of forming a coating layer on a surface of the silica particle by adding 0.1 to 0.5 parts by weight of a reducer including ascorbic acid or sodium borohydride (NaBH₄), 5 to 25 parts by weight of a mixture obtained by mixing a Ag ion precursor and a metallic salt in a weight ratio of 1:2 to 9, and 2000 to 3000 parts by weight of an alcohol solvent in 100 parts by weight of the silica particle to prepare a resultant, and maintaining the resultant for 3 to 4 hours at a temperature of 35 to 38° C. to induce a reaction of the resultant.

In this case, the alcohol solvent may include any one selected from the group consisting of methanol, ethanol, and propanol. The Ag ion precursor may include any one selected from the group consisting of silver nitrate, silver nitrite, and silver perchlorate. The metallic salt may include any one selected from the group consisting of nitrate, phosphate, and carbonate. In addition, metallic ion of the metallic salt may include any one selected from the group consisting of zinc (Zn), magnesium (Mg), calcium (Ca), copper (Cu), and zirconium (Zr).

The coating layer may have a thickness of 2 nm to 5 nm. An amount of an Ag-containing metallic component of the coating layer may be 2 to 9 parts by weight based on 100 parts by weight of the silica particle.

The method may further include, after the coating layer is formed, preparing a dispersion solution by dispersing the silica particle, on which the coating layer is formed, to a concentration of 1,000 to 3,000 ppm in any one solvent selected from the group consisting of 1,3-butylene glycol, glycerin, and polyethylene glycol. According to another aspect of the present invention, there is provided a topical dermatological composition including 0.0001 wt % to 0.001 wt % of a silica powder coated with an antibacterial agent, prepared by using the above-described method.

### Advantageous Effects

A silica powder coated with an antibacterial agent according to the present invention may have high antibacterial capability, and thus, topical dermatological products including the silica powder may be maintained for a long time without using a chemical antiseptic agent that may irritate the skin and cause an allergy in the human body. Even if a small amount of expensive silver (Ag) ion material is used, the silica powder has excellent antibacterial capability and stability, and thus, an inexpensive powder coated with an antibacterial agent may be prepared.

In addition, since the silica powder according to the present invention exhibits bright color, colors may be freely expressed when the silica powder is used in a composition of cosmetics. Thus, there is no limit in using the silica powder in bright-color cosmetics as well as in dark-color cosmetics.

DESCRIPTION OF THE DRAWINGS

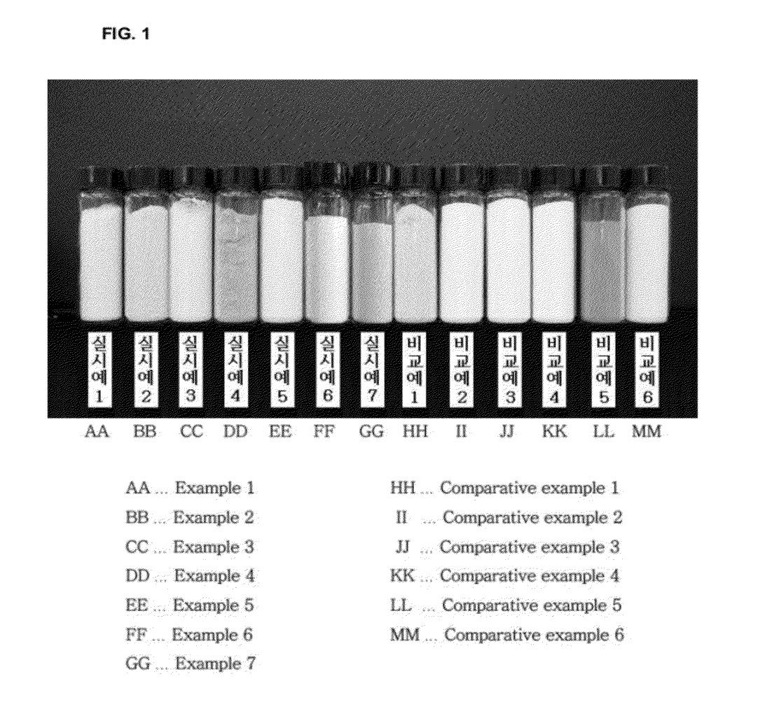

FIG. 1 is a color image of silica powders coated with antibacterial agents.

BEST MODE

According to an embodiment of the present invention, a silica powder coated with an antibacterial agent has a core/shell structure, wherein the silica powder functions as a core and a coating layer of the antibacterial agent is formed on a surface of the core to constitute a shell.

Thus, a method of manufacturing the silica powder coated with the antibacterial agent may largely include two processes that include a process of forming the silica powder functioning as the core and a process of forming the coating layer of the antibacterial agent functioning as the shell.

Minute powders that correspond to the resulting material of the process of forming the silica powder may have diameters that are distributed within a predetermined range in consideration of antibacterial properties and stability. To this end, an average diameter of the minute powders may be appropriately adjusted so as to fall within a predetermined range.

To this end, silicon alkoxide reacts in the presence of a catalyst in an alcohol solvent for 6 to 7 hours at a temperature of 35° C. to 38° C. to form silica particles. is The silicon alkoxide is used as a silica precursor. Examples of the silicon alkoxide may include, but are not limited to, tetraethylorthosilicate (TEOS), sodium silicate, or the like.

Examples of the catalyst used to form the silica powder may include aqueous ammonia, ammonium bicarbonate, triethanolamine, or the like. The amount of the catalyst may be 10 to 15 parts by weight based on 100 parts by weight of the silicon alkoxide. When the amount of the catalyst is outside this range, the particle sizes of the silica powder may be irregularly distributed.

Examples of the alcohol solvent used to form the silica powder may include methanol, ethanol, propanol, or the like. The amount of the alcohol solvent may be 700 to 800 parts by weight based on 100 parts by weight of the silicon alkoxide. When the amount of the alcohol solvent is outside this range, the viscosity of a reaction solution is too high or low.

The types and amounts of the materials used to form the silica powder are important and also reaction conditions, that is, a reaction time and a reaction temperature may be appropriately controlled.

The reaction may proceed for a reaction time of 6 to 7 hours. When the reaction proceeds for a reaction time less than 6 hours, the reaction does not proceed sufficiently.

When the reaction proceeds for a reaction time greater than 7 hours, since secondary particles are generated after primary particles are generated, the particle sizes of the silica powder may be irregularly distributed.

The reaction may proceed at a reaction temperature of 35° C. to 38° C. . When the reaction proceeds at a reaction temperature less than 35° C., the reaction time is increased.

When the reaction proceeds at a reaction temperature greater than 38° C., particles of the silica powder may not be uniformly formed.

The silica particles prepared by using the above-described method have an average diameter of 100 nm to 150 nm. When the average diameter of the silica powder is less than 100 nm, the silica powder may penetrate into the human body including skin to generate toxicity. When the average diameter of the silica powder is greater than 150 nm, a surface area of the silica powder is reduced, and thus, the antibacterial properties or the like may reduce.

With regard to the silica powder prepared by the above-described process, a shell layer coated with an antibacterial agent is formed in a subsequent process. That is, the subsequent process means that the silica particle obtained in the above-described process, a silver (Ag) ion precursor, and a metallic salt are mixed together in the alcohol solvent, and then a reducer is added such that the Ag ion precursor is reduced to form the shell layer on surfaces of the silica particles together with the metallic salt.

In the process of forming the shell layer, the reaction proceeds for 3 to 4 hours at a temperature of 35° C. to 38° C. in order to reduce the Ag ion precursor and to sufficiently coat the metallic salt on the surfaces of the silica particles.

When the reaction time is less than 3 hours, the reaction may not sufficiently proceed, and thus, a sufficient amount of the coating layer may not be formed. When the reaction time is greater than 4 hours, the reaction may sufficiently occur already before the reaction time elapses, economical features may be reduced.

Examples of the Ag ion precursor used to form the shell layer may include silver nitrate, silver nitrite, silver perchlorate, or the like. Examples of the metallic salt may include nitrate, phosphate, carbonate, or the like. Examples of metallic ion of the metallic salt may include zinc (Zn), magnesium (Mg), calcium (Ca), copper (Cu), zirconium (Zr), or the like.

The total amount of the Ag ion precursor and the metallic salt that are mixed in the alcohol solvent may be 5 to 25 parts by weight based on 100 parts by weight of the silica powder. When the total amount is less than 5 parts by weight, a sufficient amount of the coating layer may not be formed. When the total amount is greater than 25 parts by weight, the coating layer is too thick or a large amount of unreactive materials remain, and thus, economical features may be reduced.

With regard to a mixture ratio of the Ag ion precursor and the metallic salt, the weight of the metallic salt may be 2 to 9 times greater than that of the Ag ion precursor. Examples of the alcohol solvent used to form the coating layer may include methanol, ethanol, propanol, or the like. The amount of the alcohol solvent may be 2,000 to 3,000 parts by weight based on 100 parts by weight of the silica powder.

Examples of the reducer that is used to reduce the Ag ion precursor during the formation of the coating layer may be a general reducer such as ascorbic acid, sodium borohydride (NaBH₄), or the like. The amount of the reducer may be 0.1 to 0.5 parts by weight based on 100 parts by weight of the silica powder.

After the process of forming the coating layer of the antibacterial agent is completed to obtain a product, impurities and unreactive materials may be removed by washing, drying, and heat-treating the product. Through the above-described process of forming the coating layer of the antibacterial agent, the Ag and metal coating layer may be uniformly formed on the surface of the silica powder and the coating layer may have a thickness of about 2 to about 5 nm.

The coating layer may be formed as a layer having a continuous and uniform shape, or alternatively, may be configured in such a way that Ag and metal particles may be combined in the form of particles that are aggregated on the surface of the silica powder.

Through the above-described process, 2 to 9 parts by weight of Ag and metallic components of the shell layer are coated on 100 parts by weight of the silica powder functioning as a core.

As described above, since relatively inexpensive metallic salts such as Zn, Mg, Ca, Cu, Zr, or the like in addition to an expensive Ag precursor such as silver nitrate, silver nitrite, silver perchlorate, or the like may be mixed and used, silica particles coated with Ag and metallic salt, which has excellent antibacterial properties and stability, may be manufactured at low manufacturing costs.

The metallic component such as Zn, Mg, Ca, Cu, Zr, or the like coated on the surfaces of the silica particles exhibits a bright ivory color compared to Ag component, and thus, the silica particle coated with Ag and metallic salt exhibit a brighter ivory color than a silica powder coated with an Ag component only.

In addition, by restricting the size of the silica powder constituting a core to a predetermined range, the silica powder may be appropriately used in a field requiring stability and antibacterial properties, such as a topical dermatological composition, or the like.

It is required to form a dispersion solution having an appropriate concentration in order to appropriately provide antibacterial properties and stability to the silica powder is coated with the antibacterial agent. That is, the silica powder coated with the antibacterial agent may be formed in the dispersion solution so as to fall within a range, for example, 1,000 ppm to 3,000 ppm.

Examples of a dispersion solvent used to form the dispersion solution may include, but are not limited to, 1,3-butylene glycol, glycerin, polyethylene glycol, or the like.

As described above, the silica powder coated with the antibacterial agent according to the present invention may be used in various topical dermatological compositions. In addition, the silica powder coated with antibacterial agent may prevent the topical dermatological compositions from being spoilt due to its excellent antibacterial properties and stability, thereby increasing storage and maintenance capabilities of the topical dermatological compositions.

The amount of the silica powder coated with the antibacterial agent of the topical dermatological composition may be 0.0001 to 0.001 wt % with respect to the total amount of the topical dermatological composition.

In addition, the topical dermatological composition includes components that are commonly used, in addition to the silica powder. For example, the topical dermatological composition may include a general additive and support, such as a stabilizer, a solubilizer, vitamin, pigment, and flavoring.

According to an embodiment of the present invention, the topical dermatological composition including the silica powder coated with the antibacterial agent may be manufactured in any type of formulations as long as the formulations are commonly used in the art. For example, the topical dermatological composition may be formulated in a type of, but is not limited to, solution, suspension, emulsion, paste, gel, cream, lotion, powder, soap, surfactant-containing cleanser, oil, powder foundation, emulsion foundation, wax foundation, spray, or the like.

When the silica powder coated with the antibacterial agent is applied to a cosmetic material as a component of the topical dermatological composition, metallic components coated on a surface of the silica powder is oxidized to exhibit white or ivory color, thereby brightening the color of compositions of the cosmetic material.

### MODE OF THE INVENTION

Hereinafter, the present invention will be described in detail with reference to the following examples, comparative examples, and test examples.

However, it will be understood by those of ordinary skill in the art that these examples are not intended to limit the purpose and scope of the present invention and various changes in form and details may be made therein without departing from the spirit and scope of the present invention.

### Example 1

1 L of 95% ethanol as a solvent was put in a 3L 4-necked reactor, 104.17 g of tetraethylorthosilicate (TEOS) was added dropwise to the reactor, and was stirred for 30 minutes such that the solution was completely mixed. Then, 9.01 g of distilled water was put in the reactor and was stirred again for 30 minutes.

Then, 13.6 g of aqueous ammonia as a catalyst was added dropwise to the reactor. While the solution was stirred, a temperature of the reactor was increased to a temperature of 36° C. . Then, reaction proceeded for 6 six hours. The reactant was washed with water, was vacuum-filtered, and was dried for 12 hours in a hot air drier at a temperature of 120° C. to prepare 30 g of minute silica powders having an average diameter of 110 nm.

Then, 1 L of 95% ethanol was put in a 3L 4-necked reactor. While 30 g of the minute silica particles were dispersed and stirred, 9.87 g of aqueous nitrate salt solution obtained by dissolving 0.53 g of silver nitrate and 2.76 g of zinc nitrate in 6.58 g of purified water was slowly added dropwise to the reactor, and then was stirred for 3 hours. In addition, 0.04 g of sodium borohydride (NaBH₄) as a reducer was put in the reactor and then was stirred for 3 hours at a temperature of 36° C.

Then, the reactant was washed with 1 L of distilled water and was vacuum-filtered to obtain a filtered cake. The obtained filtered cake was dispersed in distilled water again, was vacuum-filtered, and was dried for 12 hours in a hot air drier at a temperature of 120° C. . Then, a temperature was gradually increased to a temperature of 300 ° C. and heat-treatment was performed for 4 hours to remove impurities and unreactive materials to prepare 31.46 g of silica powder on which a Ag and Zn coating layer was formed to a is thickness of 3 nm.

### Example 2

31.23 g of silica powder containing a Ag and Mg coating layer was prepared in the same manner as in Example 1, except that 8.79 g of aqueous nitrate salt solution obtained by dissolving 0.53 g of silver nitrate and 2.40 g of magnesium nitrate in 5.86 g of purified water was added dropwise to a silica particle dispersion solution to prepare the Ag and Mg coating layer, instead of the Ag and Zn coating layer formed on the silica particle of Example 1.

### Example 3

31.01 g of silica powder containing a Ag and Ca coating layer was prepared in the same manner as in Example 1, except that 8.16 g of aqueous nitrate salt solution obtained by dissolving 0.53 g of silver nitrate and 2.19 g of calcium nitrate in 5.44 g of purified water was added dropwise to a silica particle dispersion solution to prepare the Ag and Ca coating layer, instead of the Ag and Zn coating layer formed on the silica particle of Example 1.

### Example 4

31.32 g of silica powder containing a Ag and Cu coating layer was prepared in the same manner as in Example 1, except that 8.04 g of aqueous nitrate salt solution obtained by dissolving 0.53 g of silver nitrate and 2.15 g of copper nitrate in 5.36 g of purified water was added dropwise to prepare the Ag and Cu coating layer, instead of the Ag and Zn coating layer formed on the silica particle of Example 1.

### Example 5

34.21 g of silica powder containing a Ag and Zr coating layer was prepared in the same manner as in Example 1, except that 11.01 g of aqueous nitrate salt solution obtained by dissolving 0.53 g of silver nitrate and 3.14 g of zirconium nitrate in 7.34 g of purified water was added dropwise to prepare the Ag and Zr coating layer, instead of the Ag and Zn coating layer formed on the silica particle of Example 1.

### Example 6

31.54 g of silica powder containing a Ag and Zn coating layer was prepared in the same manner as in Example 1, except that 9.56 g of aqueous metallic salt solution obtained by dissolving 0.53 g of silver nitrate and 4.25 g of zinc phosphate in 4.78 g of purified water was added dropwise to prepare the Ag and Zn coating layer, instead of the Ag and Zn coating layer formed on the silica particle of Example 1.

### Example 7

31.43 g of silica powder containing a Ag and Zn coating layer was prepared in the same manner as in Example 1, except that 3.38 g of aqueous metallic salt solution obtained by dissolving 0.53 g of silver nitrate and 1.16 g of zinc carbonate in 1.69 g of purified water was added dropwise to prepare the Ag and Zn coating layer, instead of the Ag and Zn coating layer formed on the silica particle of Example 1.

### Comparative Example 1

30.7 g of silica powder containing a Ag coating layer was prepared in the same manner as in Example 1, except that 4.74 g of aqueous silver nitrate solution obtained by dissolving 1.58 g of silver nitrate in 3.16 g of purified water and 0.03 g of NaBH₄ as a reducer were added, instead of the Ag and Zn coating layer formed on the silica particle of Example 1.

### Comparative Example 2

31.1 g of silica powder containing a Zn coating layer was prepared in the same manner as in Example 1, except that 8.28 g of aqueous zinc nitrate solution obtained by dissolving 2.76 g of zinc nitrate in 5.52 g of purified water and 0.03 g of NaBH₄ as a reducer were added, instead of the Ag and Zn coating layer formed on the silica particle of Example 1.

### Comparative Example 3

31.0 g of silica powder containing a Mg coating layer was prepared in the same manner as in Example 1, except that 7.20 g of aqueous magnesium nitrate solution obtained by dissolving 2.40 g of magnesium nitrate in 4.80 g of purified water and 0.03 g of NaBH₄ as a reducer were added, instead of the Ag and Zn coating layer formed on the silica particle of Example 1.

### Comparative Example 4

30.9 g of silica powder containing a Ca coating layer was prepared in the same manner as in Example 1, except that 6.57 g of aqueous calcium nitrate solution obtained by dissolving 2.19 g of calcium nitrate in 4.38 g of purified water and 0.03 g of NaBH₄ as a reducer were added, instead of the Ag and Zn coating layer formed on the silica particle of Example 1.

### Comparative Example 5

31.0 g of silica nano powder containing a Cu coating layer was prepared in the same manner as in Example 1, except that 5.31 g of aqueous copper nitrate solution obtained by dissolving 2.15 g of copper nitrate in 3.16 g of purified water and 0.03 g of NaBH₄ as a reducer were added, instead of the Ag and Zn coating layer formed on the silica particle of Example 1.

### Comparative Example 6

32.1 g of silica powder containing a Zr coating layer was prepared in the same manner as in Example 1, except that 9.42 g of aqueous zirconium nitrate solution obtained by dissolving 3.14 of zirconium nitrate in 6.28 g of purified water and 0.03 g of NaBH₄ as a reducer were added, instead of the Ag and Zn coating layer formed on the silica particle of Example 1.

### Test Example 1

### Analysis of Amount of Metallic Components

To check the amounts of metallic components of the silica powders prepared in

Examples 1 to 7 and Comparative Examples 1 to 6, the amounts were analyzed by using inductively coupled plasma (ICP, OPTMA 5300DV, PerkinElmer, USA) and the analysis results were shown in Table 1 below.

TABLE 1
Analysis result of metallic component of silica powder

| | Ag | Zn | Mg | Ca | Cu | Zr |
|---|---|---|---|---|---|---|
| | concen- | concen- | concen- | concen- | concen- | concen- |
| | tration | tration | tration | tration | tration | tration |
| | (wt %) | (wt %) | (wt %) | (wt %) | (wt %) | (wt %) |
| Example 1 | 0.8% | 1.5% | — | — | — | — |
| Example 2 | 0.9% | — | 1.2% | — | — | — |
| Example 3 | 0.8% | — | — | 1.0% | — | — |
| Example 4 | 0.8% | — | — | — | 1.2% | — |
| Example 5 | 0.8% | — | — | — | — | 1.4% |
| Example 6 | 0.8% | 1.4% | — | — | — | — |
| Example 7 | 0.8% | 1.3% | — | — | — | — |
| Comparative | 2.3% | — | — | — | — | — |
| Example 1 | | | | | | |
| Comparative | — | 3.8% | — | — | — | — |
| Example 2 | | | | | | |
| Comparative | — | — | 3.6% | — | — | — |
| Example 3 | | | | | | |
| Comparative | — | — | — | 3.2% | — | — |
| Example 4 | | | | | | |
| Comparative | — | — | — | — | 2.9% | — |
| Example 5 | | | | | | |
| Comparative | — | — | — | — | — | 4.3% |
| Example 6 | | | | | | |

### Test Example 2

### Antibacterial Capability Test

In order to evaluate the antibacterial capabilities of the powders prepared in Examples 1 to 7 and Comparative Examples 1 to 6 and the dispersion solutions including the powders, minimum inhibitory concentration (MIC) was checked by measuring inhibitory capability for each concentration.

The dispersion solution prepared by dispersing the powder to a concentration of 3000 ppm in glycerin was used. Pseudomonas aeruginosa (ATCC 9027), staphylococcus aureus (ATCC 6538), escherichia coli (ATCC 8739), candida albicans (ATCC 10231), and aspergillus niger (ATCC 16404) were each used as test strain.

With regard to strain culture, bacteria were inoculated to tryptic soy broth (TSA) and were cultured for 24 hours at a temperature of 37° C. In addition, yeast and filamentous fungi were inoculated to potato dextrose broth (PDA) and were cultured 2 days to 3 days at a temperature of 25° C.

Then, for the respective samples, that is, the respective powders prepared in Examples 1 to 7 and Comparative Examples 1 to 6, and methylparaben and ethylparaben that are widely used as an antiseptic agent of a cosmetic material as a control group were each diluted to an appropriate concentration in nutrient broth or PDA, and 100 μl of the diluted solution for each respective sample concentration was put in a 96-well plate. Then, with regard to bacteria, bacteria were diluted to a final concentration of about 2×10⁶ CFU/ml to about 4×10⁶CFU/ml. With regard to filamentous fungi, filamentous fungi were diluted to a final concentration of about 2×10⁵ CFU/ml to about 4×10⁵CFU/ml. Then, 100 μl of the diluted solution was put in a 96-well plate and was well mixed with a test sample. With regard to bacteria, the mixture was cultured for 24 hours at a temperature of 37° C. With regard to filamentous fungi, the mixture was cultured for 48 hours at a temperature of 37° C. Then, the turbidity of the well plates including bacteria and filamentous fungi were each compared with the turbidity of a well plate that does not include any sample to check whether bacteria were grown.

The lowest minimum concentrations at which growth of bacteria were inhibited with no turbidity in the well plates were measured as a minimum inhibitory concentration (MIC). The results are shown in Table 2 below.

TABLE 2
Minimum Inhibitory Concentration (MIC) Result

| | | Minimum Inhibitory Concentration (MIC) (powder: ppm/ | | | | |
|---|---|---|---|---|---|---|
| | | Dispersion solution: %) | | | | |
| Division | | E. coli | S. aureus | P. aeruginosa | C. albicans | A. niger |
| Example 1 | Dispersion | 12.5 | 12.5 | 25 | 100 | 100 |
| | Dispersion | 0.25% | 0.25% | 0.25% | 0.5% | 1.0% |
| | solution | | | | | |
| Example 2 | Dispersion | 25 | 50 | 25 | 100 | 200 |
| | Dispersion | 0.5% | 1.0% | 1.0% | 2.0% | 2.0% |
| | solution | | | | | |
| Example 3 | Dispersion | 25 | 25 | 50 | 200 | 200 |
| | Dispersion | 1.0% | 0.5% | 0.5% | 2.0% | 2.0% |
| | solution | | | | | |
| Example 4 | Dispersion | 100 | 200 | 100 | 100 | 100 |
| | Dispersion | 1.0% | 1.0% | 1.0% | 2.0% | 1.0% |
| | solution | | | | | |
| Example 5 | Dispersion | 200 | 200 | 200 | 400 | 200 |
| | Dispersion | 4.0% | 4.0% | 4.0% | 8.0% | 4.0% |
| | solution | | | | | |
| Example 6 | Dispersion | 25 | 12.5 | 25 | 100 | 100 |
| | Dispersion | 0.5% | 0.25% | 0.25% | 1.0% | 1.0% |
| | solution | | | | | |
| Example 7 | Dispersion | 25 | 25 | 25 | 100 | 200 |
| | Dispersion | 0.5% | 0.25% | 0.5% | 1.0% | 2.0% |
| | solution | | | | | |
| Comparative | Dispersion | 50 | 50 | 50 | 200 | 400 |
| Example 1 | Dispersion | 1.0% | 1.0% | 0.5% | 2.0% | 4.0% |
| | solution | | | | | |
| Comparative | Dispersion | 400< | 400< | 400< | 800< | 800< |
| Example 2 | Dispersion | 8.0%< | 8.0%< | 8.0%< | 16%< | 16%< |
| | solution | | | | | |
| Comparative | Dispersion | 400< | 400< | 400< | 800< | 800< |
| Example 3 | Dispersion | 8.0%< | 8.0%< | 8.0%< | 16%< | 16%< |
| | solution | | | | | |
| Comparative | Dispersion | 400< | 400< | 400< | 800< | 800< |
| Example 4 | Dispersion | 8.0%< | 8.0%< | 8.0%< | 16%< | 16%< |
| | solution | | | | | |
| Comparative | Dispersion | 400 | 800 | 400 | 400 | 400 |
| Example 5 | Dispersion | 4.0% | 8.0% | 4.0% | 4.0% | 8.0% |
| | solution | | | | | |
| Comparative | Dispersion | 400< | 400< | 400< | 800< | 800< |
| Example 6 | Dispersion | 8.0%< | 8.0%< | 8.0%< | 16%< | 16%< |
| | solution | | | | | |
| control | Methylparaben | 800 | 800 | 1000 | 1000 | 600 |
| group | Ethylparaben | 600 | 500 | 800 | 800 | 400 |

As shown in Table 2 above, the powders prepared in Examples 1 through 7 have higher antibacterial capabilities than those of the powders prepared in Comparative Examples 1 through 6 and have much higher antibacterial capabilities than those of the paraben-based compounds suggested as a control group.

That is, when a powder is coated with Zn, Mg, Ca, Cu, or Zr alone, the antibacterial capability of the powder is low. However, when a powder is coated with Zn, Mg, Ca, Cu, or Zr together with Ag, even if a smaller amount of Ag is used than a case where Ag is coated alone, the powder has the same antibacterial capability as the case where Ag is coated alone.

Orders of the antibacterial capabilities of metal ions have been reported in the academic world as follows: Ag>Hg>Cu>Cd>Cr>Pb>Co>Au>Zn>Fe>Mn>Mo>Sn. In addition, it is known that the antibacterial capability of Ag is remarkably high. Even though it is known that Zn has antibacterial capability, the antibacterial capability is very low, as shown in Table 2 above, and thus, it is not appropriate to industrially use Zn.

Zn, Mg, Ca, Cu, or Zr itself does not have antibacterial capability. However, these ions are vital elements for growth of bacteria or filamentous fungi, these ions are combined with antibacterial materials to serve as an attractant of bacteria. Thus, it is determined that, when these ions has increased dispersion properties and solubility with respect to water and are combined with a strong antibacterial material such as Ag, the antibacterial capability of the antibacterial material is maximized.

Accordingly, when the powder including the silica particle prepared in each of Examples 1 to 7 coated with Ag and Mg, Ca, An, Cu, or Zr and a dispersion solution is mixed with a cosmetic composition, various topical dermatological products may be maintained for a long time without using a chemical antiseptic agent that may irritate the skin and cause an allergy in the human body.

### Test Example 3

### Chromaticity Comparison

A color image of the silica powders coated with an antibacterial agent, which are prepared in Examples 1 to 7 and Comparative Examples 1 through 6, is shown in FIG. 1.

A spectrophotometer (CM-3500d, Konika milota, Japan) was used to accurately measure chromatic aberrations of colors. The measurement results are shown in Table 3 below.

TABLE 3
Chromaticity comparison result

| | L^{note1)} | a^{note 2)} | b^{note 3)} |
|---|---|---|---|
| Example 1 | 94.16 | −0.49 | 10.43 |
| Example 2 | 86.72 | 1.43 | 18.52 |
| Example 3 | 93.58 | 0.34 | 13.37 |
| Example 4 | 67.33 | −2.33 | 3.34 |
| Example 5 | 94.26 | 0.89 | 10.75 |
| Example 6 | 96.04 | 0.07 | 0.85 |
| Example 7 | 87.06 | 1.22 | 16.59 |
| Comparative | 81.20 | 2.69 | 20.88 |
| Example 1 | | | |
| Comparative | 96.00 | 0.26 | 2.12 |
| Example 2 | | | |
| Comparative | 98.21 | 0.03 | 1.92 |
| Example 3 | | | |
| Comparative | 98.99 | 0.03 | 1.06 |
| Example 4 | | | |
| Comparative | 55.66 | −1.18 | −9.07 |
| Example 5 | | | |
| Comparative | 98.57 | 0.17 | 1.06 |
| Example 6 | | | |

^{note 1)}L = 0: black, L = 100: white
^{note 2)}a = positive number: red, a = negative number: green
^{note 3)}b = positive number: yellow, b = negative number: blue

As shown in FIG. 1 and Table 3 above, the silica powder containing the Cu coating layer of Comparative Example 5 exhibits the darkest color, the silica powder containing the Ag coating layer of Comparative Example 1 exhibits a relatively dark color, and the silica powder containing the Zn, Ma, Ca, and Zr coating layers of Examples 1, 2, 3, 5, 6, and 7 and Comparative Examples 2, 3, 4, and 6 exhibit bright colors.

Via the chromaticity comparison result, when silica particles are coated with Ag and Zn, Ag and Mg, Ag and Ca, and Ag and Zr, the silica particles exhibit relatively bright colors compared to a case where a silica particle is coated with Ag only. Thus, when the powders having the bright colors are used in cosmetic material, the powders do not impose restrictions on expression of bright colors of the cosmetic material.

### Examples 8 to 11 and Comparative Examples 7 and 8

### Preparation of Lotion Base

A lotion base was prepared by using a dispersion solution obtained by dissolving the silica powder coated with the antibacterial agent of Example 1, which has an excellent capability in the above-described experiment, to a concentration of 1000 ppm in glycerin as a dispersion medium, or an existing chemical antiseptic agent. The lotion base in this experiment is an emulsion-type and had a composition as shown in Table 4 below.

TABLE 4
Composition of the emulsion-type lotion base

| Division | Component | wt % |
|---|---|---|
| Oil | cetyl alcohol | 1.0 |
| | Bee Wax | 0.5 |
| | Vaseline | 2.0 |
| | Squalene | 6.0 |
| | dimethyl-polysiloxane | 2.0 |
| Alcohol | ethanol | 5.0 |
| Humectant | glycerin | 4.0 |
| | 1,3-butylene glycol | 4.0 |
| Surfactant | POE(10) Monooleate ester | 1.0 |
| | Glycerol monostearate | 1.0 |
| | ester | |
| Mucilage | Quince Geed extract (5% | 2.0 |
| | aqueous solution) | |
| Antiseptic agent | Dispersion solution of silica | 0.1-1.0 |
| | coated with antibacterial | |
| | agent, or chemical | |
| | antiseptic agent | |
| Colorant | Dye (1% aqueous solution) | 0.001-0.003 |
| Fragrance | | 0.001-0.003 |
| Distilled water | | to 100 |

In order to prepare an aqueous part, a humectant and a colorant were added to distilled water and were heated and adjusted at a temperature of 70° C. In order to prepare an oil part, a surfactant and an antiseptic agent were added to oil and were heated and adjusted at a temperature of 70° C. The oil part was added to the aqueous part to be pre-emulsified. To the pre-emulsified, Quince Ceed Extract and ethanol were added and stirred. An emulsified particle in the resultant was uniformalized in a homogenizer. Then, Degassing, filtration and cooling was implemented to prepare a cosmetic liquid.

In a process of lotion bases of Examples 8 to 11, the dispersion solution which is produced by dispersing silica powder coated with antibacterial agent in an example 1 into the glycerin is used as a component of an antiseptic agent in the composition of emulsion-type lotion base of Table 4. And, the amount of the dispersion solution as antiseptic agent is described in Table 5 below.

In addition, it is also produced the lotion base in Comparative Example 7 including is a chemical antiseptic agent, and in Comparative Example 8 not including antiseptic agent.

TABLE 5
Amount of antiseptic agent

| | Example | Example | Example | Example | Comparative | Comparative |
|---|---|---|---|---|---|---|
| Component | 8 | 9 | 10 | 11 | Example 7 | Example 8 |
| Dispersion | 0.1 wt % | — | — | — | — | — |
| solution | — | 0.3 wt % | — | — | — | |
| | — | | 0.5 wt % | — | — | |
| | — | — | | 1.0 wt % | — | |
| methylparaben | — | — | — | — | 0.4 wt % | — |
| propylparaben | — | — | — | — | 0.2 wt % | — |
| phenoxy | — | — | — | — | 0.4 wt % | — |
| ethanol | | | | | | |
| Distilled | — | — | — | — | — | 1.0 wt % |
| water | | | | | | |

### Test Example 4

### Test of Antiseptic Capability in Formulation

In order to check antiseptic capability in formulation including a dispersion solution of silica coated with the antibacterial agent, the test of the antiseptic activities of the lotion bases prepared in Examples 8 to 11 and Comparative Examples 7 and 8 was performed.

The test of antiseptic effectiveness is a test for determining a minimum concentration of an antiseptic agent for no skin irritation and stable maintenance of a product and is very important for the stability of products and consumer adoption. As a method of measuring the antiseptic capabilities of cosmetics, a USP method and a Cosmetic, Toiletry and Fragrance Association (CTFA) are widely used. These methods use a test method of a microorganism guideline of the CTFA.

The same strain as in Test Example 3 was used as a test strain. 3 types of bacteria were inoculated to TSA and yeast and filamentous fungi were inoculated to PDA. is The bacteria and the yeast and filamentous fungi were cultured for 24 hours to 72 hours in a culture aquarium at a temperature 37° C. and a culture aquarium at a temperature 25° C., respectively and were suspended with 0.8% physiological saline such that the bacteria had a concentration of about 1×10⁸ CFU/ml and the yeast and filamentous fungi had a concentration of about 1×10⁷CFU/ml

200 μl of the suspension including the 3 types of bacteria, and 200 μl of the suspension including the yeast and filamentous fungi were each inoculated to 20 g of a sample, that is, each of the lotion bases prepared in Examples 8 through 11 and Comparative Examples 7 and 8. In this case, a final concentration of the bacteria was 1x10⁶ CFU/g and a final concentration of the yeast and filamentous fungi was 1×10⁵ CFU/g. Then, the samples were each maintained at a temperature of 25° C. 1 g of each sample was extracted under sterilization over a period of 1, 2, 3, 7, 14, 21, and 28 days and was appropriately diluted with a diluted solution. The bacteria were inoculated to TSA and the filamentous fungi were inoculated to PDA. The bacteria and the filamentous fungi were cultured for 24 hours to 72 hours at temperatures of 37° C. and 25° C., respectively and were counted.

With regard to the bacteria, the antiseptic effectiveness requires that the number of strains to be reduced by Log 3 or more seven days after the bacteria are inoculated and not to be increased during the test. With regard to the filamentous fungi, the antiseptic effectiveness requires that the number of strains to be reduced by Log 1 or more seven days after the filamentous fungi are inoculated and not to be increased during the test.

The results are shown in Tables 6 and 7 below.

TABLE 6
Measurement result of antiseptic capability against bacteria

| | Viable Cell (CFU/g) | | | | |
|---|---|---|---|---|---|
| Division | 3 days | 1 week | 2 weeks | 3 weeks | 4 weeks |
| Example 8 | 2.52 | 2.90 | — | — | — |
| Example 9 | 3.00 | 3.00 | 4.00 | 5.00 | 5.00 |
| Example 10 | 3.00 | 3.00 | 4.00 | 5.00 | 5.00 |
| Example 11 | 3.00 | 3.00 | 4.00 | 5.00 | 5.00 |
| Comparative | 3.70 | 4.00 | 5.00 | 5.00 | 5.00 |
| Example 7 | | | | | |
| Comparative | −0.30 | −1.30 | — | — | — |
| Example 8 | | | | | |

TABLE 7
Measurement result of antiseptic capability against filamentous fungi

| | Viable Cell (CFU/g) | | | | |
|---|---|---|---|---|---|
| Division | 3 days | 1 week | 2 weeks | 3 weeks | 4 weeks |
| Example 8 | 0.78 | 0.85 | — | — | |
| Example 9 | 2.00 | 2.30 | 3.00 | 4.00 | 4.00 |
| Example 10 | 2.70 | 3.00 | 4.00 | 4.00 | 4.00 |
| Example 11 | 3.00 | 3.00 | 4.00 | 4.00 | 4.00 |
| Comparative | 2.70 | 3.00 | 4.00 | 4.00 | 4.00 |
| Example 7 | | | | | |
| Comparative | −1.00 | −1.50 | — | — | — |
| Example 8 | | | | | |

As shown in Table 6 and 7 above, it is confirmed that the lotion bases prepared in Examples 8 to 11 have the same antiseptic activities for extinction of bacteria as in Comparative Example 7 using an existing chemical antiseptic agent. A case of filamentous fungi has the same result as in the case of bacteria.

### INDUSTRIAL APPLICABILITY

As described above, since a silica powder coated with an antibacterial agent according to the present invention may have high antibacterial capability, even if a small amount of expensive silver (Ag) is used, the silica powder may be prepared at low manufacturing cost, and thus, may be widely used in many industrial fields.

Since topical dermatological products including the silica powder may be maintained for a long time without using a chemical antiseptic agent that may irritate the skin and cause an allergy in the human body, there is no limit in using the silica powder. is In addition, since the silica powder according to the present invention exhibits bright color, colors may be freely expressed. Thus, the silica powder may be used to express bright colors as well as dark colors.

1. A method of manufacturing a silica powder coated with an antibacterial agent, the method comprising:
a first operation of forming silica particles with an average diameter of 100 nm to 150 nm by mixing 10 to 15 parts by weight of any one catalyst selected from the group consisting of aqueous ammonia, ammonium bicarbonate, and triethanolamine and 700 to 800 parts by weight of an alcohol solvent in 100 parts by weight of silicon alkoxide to prepare a resultant, and maintaining the resultant for 6 to 7 hours at a temperature of 35 to 38° C. to induce a reaction of the resultant; and
a second operation of forming a coating layer on a surface of the silica particle by adding 0.1 to 0.5 parts by weight of a reducer comprising ascorbic acid or sodium borohydride (NaBH₄), 5 to 25 parts by weight of a mixture obtained by mixing a Ag ion precursor and a metallic salt in a weight ratio. of 1:2 to 9, and 2000 to 3000 parts by weight of an alcohol solvent in 100 parts by weight of the silica particle to prepare a resultant, and maintaining the resultant for 3 to 4 hours at a temperature of 35 to 38 to induce a reaction of the resultant.

2. The method of claim 1, wherein the alcohol solvent comprises any one selected from the group consisting of methanol, ethanol, and propanol.

3. The method of claim 1, wherein the Ag ion precursor comprises any one selected from the group consisting of silver nitrate, silver nitrite, and silver perchlorate.

4. The method of claim 1, wherein the metallic salt comprises any one selected from the group consisting of nitrate, phosphate, and carbonate.

5. The method of claim 1, wherein metallic ion of the metallic salt comprises any one selected from the group consisting of zinc (Zn), magnesium (Mg), calcium (Ca), copper (Cu), and zirconium (Zr).

6. The method of claim 1, wherein the coating layer has a thickness of 2 nm to 5 nm.

7. The method of claim 1, wherein an amount of an Ag-containing metallic component of the coating layer is 2 to 9 parts by weight based on 100 parts by weight of the silica particle.

8. The method of claim 1, further comprising: after the coating layer is formed, preparing a dispersion solution by dispersing the silica particle, on which the coating layer is formed, to a concentration of 1,000 to 3,000 ppm in any one solvent selected from the group consisting of 1,3-butylene glycol, glycerin, and polyethylene glycol.

9. A topical dermatological composition comprising 0.0001 wt % to 0.001 wt % of a silica powder coated with an antibacterial agent, prepared by using the method of claim 1.
